(19) United States
(12) Patent Application Publication
(10) Pub. No.: US 2025/0295342 A1
(43) Pub. Date: Sep. 25, 2025

(54) COMPACT AND CONCEALED ELECTROENCEPHALOGRAPHY HEADBAND

(71) Applicant: NeuroChamp, Peachtree City, GA (US)

(72) Inventors: Caitlin Patricia van Zyl, Peachtree City, GA (US); Jacqueline Frances van Zyl, Peachtree City, GA (US); Meg Claire Langwick Weaver, Peachtree City, GA (US)

(21) Appl. No.: 19/088,263

(22) Filed: Mar. 24, 2025

Related U.S. Application Data
(60) Provisional application No. 63/569,209, filed on Mar. 24, 2024.

(51) Int. Cl. A61B 5/256; A61B 5/00; A61B 5/372

(52) U.S. Cl. CPC A61B 5/256; A61B 5/0006; A61B 5/372; A61B 5/4094; A61B 5/6814; A61B 5/6831

(57) ABSTRACT

A compact and concealed electroencephalography (EEG) headband includes a main continuous band spanning across central, frontal, parietal and temporal lobes of a brain. The headband has comb pieces located at evenly spaced distances along a top of the main continuous band. Each comb piece has a triangular shape to facilitate electrode-scalp contact and houses a dry EEG electrode. A grounding electrode is located on a bottom of the main continuous band, contacting a mastoid bone. Stability comb pieces are located along the main continuous band to hold it flush against a scalp. Electronics and wiring are embedded in the main continuous band.

### CROSS-REFERENCE TO RELATED APPLICATIONS

This application claims priority to U.S. Application No. 63/569,209, titled COMPACT AND CONCEALED ELECTROENCEPHALOGRAPHY HEADBAND, filed Mar. 24, 2024, which is hereby incorporated by reference in its entirety.

### TECHNICAL FIELD

The present disclosure relates to electroencephalography (EEG) devices, and more particularly to a compact and concealed EEG headband for non-invasive seizure monitoring.

### BACKGROUND

Electroencephalography (EEG) is a widely used method for monitoring brain activity, particularly in the diagnosis and management of neurological conditions such as epilepsy. Traditional EEG systems often involve bulky equipment and numerous electrodes placed on the scalp, which can be uncomfortable and impractical for long-term, continuous monitoring outside of clinical settings. While EEG technology has advanced significantly, there remains a need for more user-friendly, discreet, and comfortable solutions that can provide accurate and reliable data for extended periods.

The development of wearable EEG devices has aimed to address these challenges, but many existing designs still face limitations in terms of electrode-scalp contact, user comfort, and aesthetic appeal. Reduced-montage EEG systems, which use fewer electrodes, have shown promise in detecting generalized seizures while potentially offering a more compact form factor. However, achieving consistent electrode contact through hair and maintaining stability during daily activities continue to be significant hurdles in the design of practical, long-term EEG monitoring solutions for patients requiring ongoing seizure detection and management.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

A compact and concealed electroencephalography (EEG) headband is an aspect of the described technology. This aspect includes a main continuous band spanning across frontal, parietal, and temporal lobes of a brain, a plurality of comb pieces located at evenly spaced distances along a top of the main continuous band, each comb piece comprising a triangular shape and housing a dry EEG electrode, and a grounding electrode located on a bottom of the main continuous band, the grounding electrode contacting a mastoid bone. In an embodiment, the main continuous band comprises a flexible material. In other embodiments, the main continuous band comprises an elastic material. In a further embodiment, the plurality of comb pieces comprises stability comb pieces located along the main continuous band to hold the headband flush against a scalp. In still other embodiments, the dry EEG electrodes are positioned at T3, C3, Cz, T4, and C4 locations according to a standard 10-20 EEG system, electronics and wiring are embedded in the main continuous band, the comb pieces point away from a forehead when the headband is worn, and the electronics comprise a processor for analyzing EEG data collected by the dry EEG electrodes.

A method of using a compact and concealed electroencephalography (EEG) headband is another aspect of the described technology. This aspect includes sliding the headband from a forehead to a top of a head, the headband comprising a main continuous band and a plurality of comb pieces located along a top of the main continuous band, parting hair with tips of the comb pieces as the headband moves backward, and contacting dry EEG electrodes housed in the comb pieces with a scalp. In an embodiment, the main continuous band comprises a flexible material. In other embodiments, the main continuous band comprises an elastic material. In further embodiments, the plurality of comb pieces comprises stability comb pieces located along the main continuous band to hold the headband flush against the scalp, and the dry EEG electrodes are positioned at T3, C3, Cz, T4, and C4 locations according to a standard 10-20 EEG system. In still other embodiments, the method includes analyzing EEG data collected by the dry EEG electrodes using a processor embedded in the main continuous band, detecting generalized seizures based on the collected EEG data, and transmitting an alert upon detection of a generalized seizure.

An electroencephalography (EEG) system is another aspect of the described technology. This aspect includes a headband comprising a main continuous band and a plurality of comb pieces located along a top of the main continuous band, dry EEG electrodes housed in the comb pieces, a grounding electrode located on the main continuous band, and wiring embedded in the main continuous band connecting the dry EEG electrodes and the grounding electrode. In an embodiment, the main continuous band comprises a flexible material. In other embodiments, the main continuous band comprises an elastic material. In further embodiments, the plurality of comb pieces comprises stability comb pieces located along the main continuous band to hold the headband flush against a scalp, and the dry EEG electrodes are positioned at T3, C3, Cz, T4, and C4 locations according to a standard 10-20 EEG system. In still other embodiments, the system includes a processor embedded in the main continuous band, the processor analyzing EEG data collected by the dry EEG electrodes, detecting generalized seizures based on the collected EEG data, and transmitting an alert upon detection of a generalized seizure.

The foregoing general description of the illustrative embodiments and the following detailed description thereof are merely exemplary aspects of the teachings of this disclosure and are not restrictive.

### BRIEF DESCRIPTION OF FIGURES

These and other aspects of this technology will be readily apparent from the detailed description below and the appended drawings, which are meant to illustrate and not to limit the claims, and in which:

FIG. 1 illustrates a perspective view of one embodiment of an EEG headband, according to aspects of the present disclosure.

FIG. 2 depicts a flowchart of one embodiment of a method for using an EEG headband.

FIG. 3 illustrates an exploded view an embodiment of the EEG headband, showing a main band having sections and removable comb pieces.

FIG. 4 depicts perspective views of one embodiment of a triangular comb piece component of the EEG headband.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to compact and concealed electroencephalography (EEG) headbands for non-invasive seizure monitoring. EEG headbands in accordance with the present disclosure may provide improved comfort, discretion, and electrode-scalp contact compared to conventional EEG headsets. In some cases, the disclosed EEG headbands may allow for practical and accurate electrical readings from the brain over extended periods of time, as may be required for various medical procedures and diagnostic purposes. In some cases, long-term EEG monitoring may be used for epilepsy diagnosis and management, where continuous recordings can capture seizure activity that might not occur during shorter monitoring sessions. Sleep studies may also utilize extended EEG monitoring to assess sleep patterns and diagnose sleep disorders.

For patients with suspected intermittent neurological conditions, prolonged EEG monitoring may help capture infrequent events or subtle changes in brain activity. In some instances, extended EEG recordings may be used to evaluate the effectiveness of antiepileptic medications or to guide treatment decisions for various neurological disorders.

Intensive care units may employ continuous EEG monitoring for critically ill patients to detect non-convulsive and convulsive seizures or to assess brain function after injury. In research settings, long-term EEG recordings may be used to study brain activity patterns over time or in response to specific stimuli or interventions.

The compact and concealed EEG headband described in this disclosure may facilitate these extended monitoring procedures by providing a more comfortable and discreet option for patients, potentially improving compliance and data quality in various clinical and research applications.

The compact and concealed EEG headbands described herein may be particularly suitable for patients or users requiring constant EEG seizure monitoring outside of a hospital setting. Continuous monitoring may provide critical information to inform medical treatment and drug prescription on a patient-specific basis over the progression of a disease.

In some implementations, the disclosed EEG headbands may utilize a reduced-montage EEG design to detect generalized seizures. Generalized seizures may occur due to abnormal electrical activity across the entire brain simultaneously. Therefore, this abnormal activity may be detectable with a reduced number of EEG electrodes. The use of fewer electrodes may allow for a more compact headband design.

The EEG headbands described herein may incorporate features to improve electrode-scalp contact in the presence of hair on the scalp. Additionally, the headbands may be designed for concealment during use, allowing users to wear the devices discreetly in everyday settings. The compact and stable design may enable extended wear periods for continuous monitoring.

In some cases, the disclosed EEG headbands may provide a more user-friendly yet effective alternative to conventional EEG setups. The headbands may be designed for easy application by users without specialized training, potentially increasing accessibility of EEG monitoring technology.

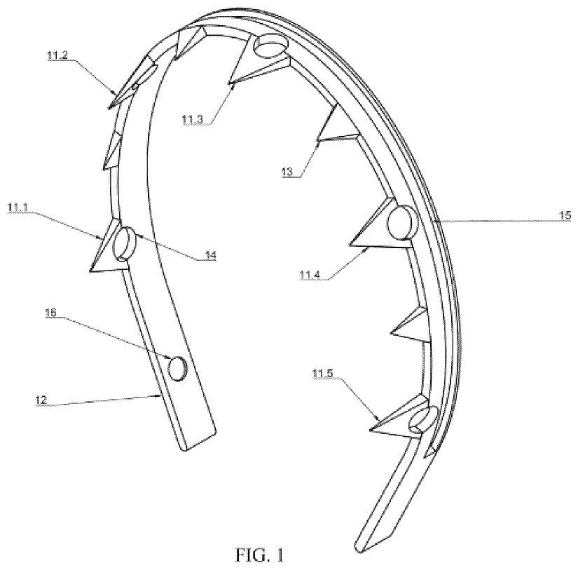

Referring now to FIG. 1, and in brief overview, the EEG headband comprises a main band 12 that may span across frontal, parietal, and temporal lobes of a brain when worn, measurement comb pieces 11 defining electrode receptacles 14, stability comb pieces 13, wiring component 15, and ground electrode receptacle 16.

Still referring to FIG. 1, and in greater detail, the main band 12 may comprise a flexible material to provide a comfortable fit for various head sizes. The main band 12 may be constructed from various flexible and durable materials such as silicone, thermoplastic elastomers, nylon-spandex blends, or other synthetic polymers that provide elasticity, comfort, and durability while allowing for the integration of electronic components and maintaining a low profile for discreet wear. In other embodiments, the main continuous band comprises an elastic material. In these other embodiments, the main band 12 may be constructed from elastic materials such as natural rubber latex, spandex fibers, or elastomeric polyurethane, which may provide stretchability and conformity to different head shapes while maintaining tension for secure electrode placement.

In some implementations, the main band 12 may comprise a rigid material molded to the head to provide a comfortable fit for various head sizes. The rigid material may be custom-formed to match the contours of an individual user's head, potentially offering a precise and stable fit. This customized rigid design may help maintain consistent electrode placement and contact pressure across different areas of the scalp.

The rigid material used for the main band 12 may include thermoplastics, such as acrylonitrile butadiene styrene (ABS) or polycarbonate, which can be heated and molded to specific head shapes. In some cases, the molding process may involve creating a 3D scan of the user's head to ensure an exact fit. The rigid structure may provide enhanced stability during movement, potentially reducing motion artifacts in the EEG recordings.

In some aspects, the rigid main band 12 may incorporate strategically placed cushioning or padding to enhance comfort at pressure points. This padding may be made from materials such as memory foam or silicone gel inserts, which can conform to the user's head shape while providing a soft interface between the rigid band and the scalp.

The rigid design may also allow for precise positioning of the electrode receptacles 14 and stability comb pieces 13, potentially improving the consistency of EEG measurements across multiple wear sessions. In some implementations, the rigid main band 12 may feature adjustable segments or modular components to accommodate changes in head size or shape over time, such as those that might occur during a child's growth.

The main band 12 may include a plurality of measurement comb pieces 11, 11.1, 11.2 (referred to, generally, as 11). In some implementations, the measurement comb pieces 11 are located at evenly spaced distances along the main band 12. In other implementations, the measurement comb pieces 11 may be positioned at various intervals along the main band 12, such as equidistant spacing, strategically spacing to conform to key EEG measurement locations, clustered in groups for enhanced stability, or arranged in an asymmetric pattern to accommodate different head shapes and sizes while maintaining optimal electrode contact and signal acquisition. The measurement comb pieces 11 may have a triangular shape designed to part the hair and allow dry EEG electrodes to rest flush against the scalp. In some cases, the mid-bases of the measurement comb pieces 11 may remain securely under the parted hair, ensuring stability and providing a slight downward force on the EEG electrodes.

Although, as shown in FIG. 1, each measurement comb piece 11 may have a triangular shape, the measurement comb pieces 11 may have shapes other than triangular. For example, the measurement comb pieces 11 may have a curved or rounded shape, which may provide a smoother interface with the scalp and potentially improve comfort during extended wear. In other aspects, the measurement comb pieces 11 may have a rectangular or square shape, which may offer increased surface area for electrode contact. The measurement comb pieces 11 may also be designed with a tapered shape, wider at the base and narrowing towards the tip, which may help in parting hair and maintaining stability. In some cases, the measurement comb pieces 11 may have a forked or multi-pronged design, potentially allowing for better hair separation and improved scalp contact. In still other implementations the measurement comb pieces 11 may incorporate a hybrid design, combining different shapes to optimize both function and comfort. For instance, they may have a rounded base transitioning to a more pointed tip. In other implementations, the measurement comb pieces 11 may have an asymmetrical shape, designed to accommodate specific head contours or hair patterns. In some aspects, the measurement comb pieces 11 may be adjustable or interchangeable, allowing users to select shapes that best suit their individual needs or preferences. The shape of the measurement comb pieces 11 may also be customized based on their specific location on the headband, potentially optimizing electrode placement and stability for different areas of the scalp. The measurement comb pieces 11 may point away from a forehead when the headband is worn, facilitating proper placement and stability.

Each measurement comb piece 11 defines a receptacle 14 for receiving a dry EEG electrode. The electrode receptacles 14 may house the dry EEG electrodes, positioning them at specific locations on the scalp for optimal EEG signal acquisition. In some cases, the dry EEG electrodes may be positioned at T3, C3, Cz, T4, and C4 locations according to a standard 10-20 EEG system, providing coverage of key brain areas for EEG monitoring.

In other implementations, the EEG headband may utilize alternative electrode positioning systems beyond the standard 10-20 system. For instance, the headband may incorporate electrode placements based on the 10-10 system, which provides a higher density of electrode locations. This system may allow for more precise spatial resolution in EEG recordings, potentially enabling more detailed mapping of brain activity.

The headband may also be designed to accommodate electrode placements according to the 10-5 system, which offers an even higher density of electrode positions. This system may be particularly useful in applications requiring very fine spatial resolution, such as source localization studies or advanced brain-computer interface applications.

In some cases, the EEG headband may be configured to use the Modified Combinatorial Nomenclature (MCN) system, which provides a standardized method for describing intermediate electrode positions. This system may allow for flexible electrode placement while maintaining consistency in naming conventions across different studies or clinical applications.

For certain specialized applications, the headband may incorporate custom electrode positioning schemes. For example, it may use dense array configurations with 64, 128, or even 256 electrode positions for high-resolution EEG studies. Alternatively, it may employ specific electrode arrangements optimized for particular brain regions of interest, such as frontal lobe activity monitoring or temporal lobe epilepsy studies.

In some implementations, the headband may be designed with adjustable or modular electrode positioning, allowing users or clinicians to modify the electrode layout based on specific diagnostic or research needs. This flexibility may enhance the versatility of the device across various EEG applications and user populations.

The choice of electrode positioning system may depend on factors such as the intended application, required spatial resolution, and the balance between comprehensive coverage and user comfort. The compact and concealed design of the EEG headband may necessitate careful consideration of electrode placement to maximize signal quality while maintaining its discreet and user-friendly nature.

In some implementations, the main band 12 may also include stability comb pieces 13 located along the main band 12. These stability comb pieces 13 may help hold the headband flush against a scalp, ensuring consistent contact between the electrodes and the user's head.

As shown in FIG. 1, the stability comb pieces 13 may be positioned at various locations along the main band 12. In some implementations, the stability comb pieces 13 may be arranged in an alternating pattern with the measurement comb pieces 11, providing balanced support across the headband. In other aspects, the stability comb pieces 13 may be clustered in groups near areas requiring additional stability, such as the temporal regions or the back of the head. The stability comb pieces 13 may also be positioned along the edges of the main band 12, creating a secure perimeter that helps maintain the headband's shape and position.

The orientation of the stability comb pieces 13 may vary depending on their location and intended function. In some cases, they may be angled slightly inward towards the scalp to provide a gentle gripping action. Alternatively, the stability comb pieces 13 may be oriented perpendicular to the main band 12 surface, maximizing their contact area with the hair and scalp.

The stability comb pieces 13 may incorporate different geometries to optimize their stabilizing function. For example, the stability comb pieces 13 may have a rounded or dome-shaped profile, which may provide a smooth interface with the scalp and potentially improve comfort during extended wear. In other implementations, the stability comb pieces 13 may have a flattened or paddle-like shape, offering increased surface area for stability without adding significant bulk to the headband design.

The size of the stability comb pieces 13 may vary depending on their location and specific role in maintaining headband position. In some aspects, they may be smaller than the measurement comb pieces 11, allowing for more discrete placement and minimal interference with hair styling. Alternatively, the stability comb pieces 13 may be designed with a wider base and shorter height, providing a low-profile yet effective stabilizing element.

In some implementations, the stability comb pieces 13 may incorporate a multi-pronged or forked design, potentially allowing for better hair separation and improved scalp contact. This design may help distribute pressure more evenly and prevent the headband from shifting during use. The stability comb pieces 13 may also feature a textured surface or small ridges to enhance their gripping capabilities without causing discomfort.

The shape and size of the stability comb pieces 13 may be customized based on their specific location on the headband, potentially optimizing stability for different areas of the scalp. For instance, stability comb pieces 13 located near the temples may have a different profile compared to those positioned at the back of the head. In some aspects, the stability comb pieces 13 may be adjustable or interchangeable, allowing users to select configurations that best suit their individual head shape and hair type.

A wiring component 15 may be embedded within the main band 12. This wiring component 15 may connect the dry EEG electrodes to other electronic components within the headband. In some implementations, the main band 12 may also house embedded electronics, which may include a processor for analyzing EEG data collected by the dry EEG electrodes.

The wiring component 15 may terminate in a plug or connector at one end of the main band 12. This plug may allow the headband to be electrically connected to a separate wiring harness. In some implementations, the plug may be a small, low-profile connector designed to maintain the discreet nature of the headband while providing a secure and reliable electrical connection.

The wiring harness may serve as an intermediary between the headband and other external devices or processing units. This modular approach may offer several advantages. For instance, it may allow for easier maintenance and replacement of components, as the headband and wiring harness can be separated. The wiring harness may also incorporate additional features such as signal amplifiers, filters, or wireless transmission modules.

In some implementations, the EEG headband may be designed with additional features to enhance its concealability and aesthetic appeal. For example, the headband may be compatible with a detachable fashion band that can be worn over the main band 12. This fashion band may be made from various materials such as fabric, leather, or synthetic materials, and may come in different colors, patterns, or styles to suit the user's preferences. The fashion band may be designed to completely cover the main band 12 and its components, while still allowing for proper functioning of the EEG electrodes and stability comb pieces.

The detachable fashion band may utilize various attachment mechanisms to secure it to the main band 12. For instance, it may incorporate snap fasteners, hook-and-loop closures, or magnetic attachments that align with corresponding points on the main band 12. This design may allow users to easily swap out different fashion bands to match their outfits or occasions, potentially increasing the likelihood of consistent wear and use of the EEG monitoring device.

In other aspects, the EEG headband may be specifically designed to be easily concealed under existing headwear or custom-designed head coverings. The main band 12 and its components may be engineered to have a low profile, potentially allowing them to fit comfortably under various types of hats, caps, or headscarves. The materials used in the construction of the main band 12 may be selected for their flexibility and ability to conform to the shape of the head, facilitating concealment under different styles of headwear.

For users who prefer to wear hats or caps, the EEG headband may be designed with a slightly flattened profile along the top of the head. This configuration may help minimize any visible bulges or protrusions when worn under a hat. The measurement comb pieces 11 and stability comb pieces 13 may be engineered to be as low-profile as possible while still maintaining their functionality.

In some implementations, custom head coverings may be designed specifically to work in conjunction with the EEG headband. These head coverings may incorporate strategically placed padding or compartments that accommodate the various components of the headband while maintaining a natural appearance. For instance, a custom-designed beanie or skull cap may feature slightly thicker areas that align with the locations of the measurement comb pieces 11, potentially helping to disguise their presence.

The EEG headband may also be designed to work with headband-style accessories commonly worn for fashion or sports purposes. In this configuration, the main band 12 may be engineered to fit securely underneath a wider, decorative headband. The decorative headband may be designed with small openings or areas of increased flexibility that align with the electrode positions, allowing for proper scalp contact while maintaining an aesthetically pleasing appearance.

For users who wear religious or cultural head coverings, the EEG headband may be designed to be compatible with these garments. For example, it may be engineered to fit comfortably under hijabs, turbans, or other traditional head coverings without interfering with their proper wear or appearance. The materials used in the construction of the main band 12 may be selected for their breathability and comfort, potentially reducing heat buildup or discomfort during extended wear under such head coverings.

In some aspects, the EEG headband may incorporate modular design elements that allow certain components, such as the measurement comb pieces 11 or stability comb pieces 13, to be temporarily removed or flattened when wearing certain types of headwear. This feature may provide users with greater flexibility in concealing the device under different styles of head coverings or in various social situations.

The wiring component 15 and any associated connectors may be designed with extra flexibility and durability to withstand the potential stress of being worn under various types of headwear. The placement of these components may also be optimized to minimize their visibility and any potential discomfort when the headband is concealed under a hat or head covering.

The wiring harness may serve as an intermediary between the headband and other external devices or processing units. This modular approach may offer several advantages. For instance, it may allow for easier maintenance and replacement of components, as the headband and wiring harness can be separated. The wiring harness may also incorporate additional features such as signal amplifiers, filters, or wireless transmission modules.

In some aspects, the wiring harness may be designed to be worn unobtrusively, such as inside clothing or attached to a belt. The harness may include its own power source, potentially extending the operational time of the EEG system without adding bulk to the headband itself.

The connection between the headband's plug and the wiring harness may be designed for quick and easy attachment and detachment. This feature may allow users to temporarily disconnect the headband, such as when adjusting its position or during brief periods when monitoring is not required, without needing to remove the entire system.

In some implementations, the wiring harness may include multiple connection points, allowing for the simultaneous use of additional sensors or devices alongside the EEG headband. This expandability may enhance the versatility of the system, potentially enabling multi-modal monitoring or the integration of other physiological sensors.

The plug and wiring harness design may also facilitate the use of different processing or transmission modules depending on the specific application or setting. For example, a compact, battery-powered module may be used for ambulatory monitoring, while a more powerful processing unit may be connected for in-depth clinical assessments or research applications.

In some implementations, the wiring harness may be connected to a transmission circuit, enabling the data collected by the electrodes to be transmitted to external systems for analysis. This configuration may allow for real-time monitoring and processing of EEG data without requiring extensive computational capabilities within the headband itself.

The transmission circuit may incorporate various wireless communication technologies to facilitate data transfer. For instance, it may utilize Bluetooth Low Energy (BLE) protocols, which may provide efficient short-range communication with low power consumption. In other cases, the transmission circuit may employ Wi-Fi technology, potentially allowing for higher data transfer rates and longer-range connectivity.

In some aspects, the transmission circuit may include cellular communication capabilities, such as 4G or 5G connectivity. This feature may enable the EEG headband to transmit data directly to cloud-based servers or remote monitoring stations, even when the user is mobile or in areas without Wi-Fi coverage.

The transmission circuit may also incorporate data compression algorithms to optimize bandwidth usage and reduce power consumption. These algorithms may be designed to preserve the integrity of the EEG signals while minimizing the amount of data that needs to be transmitted.

In some implementations, the transmission circuit may include local storage capabilities, such as flash memory or a small solid-state drive. This local storage may serve as a buffer, temporarily storing EEG data in case of interruptions in wireless connectivity. Once the connection is reestablished, the stored data may be transmitted along with real-time data, ensuring continuity in the EEG recordings.

The transmission circuit may also include encryption modules to protect the privacy and security of the transmitted EEG data. These encryption measures may comply with relevant healthcare data protection standards, such as HIPAA in the United States or GDPR in Europe.

In some cases, the transmission circuit may be designed with multiple communication modes, allowing it to automatically switch between different transmission protocols based on availability and signal strength. This adaptive approach may help ensure consistent data transmission across various environments and usage scenarios.

The transmitted data may be received by a variety of devices or systems for analysis. For example, it may be sent to a smartphone app that provides real-time visualization of the EEG data and basic analysis capabilities. In clinical settings, the data may be transmitted directly to hospital information systems for integration with other patient data and more comprehensive analysis.

In research applications, the transmission circuit may allow for the streaming of EEG data to high-performance computing clusters or cloud-based analytics platforms. These systems may apply advanced signal processing techniques, machine learning algorithms, or other computational methods to extract meaningful insights from the EEG recordings.

The ability to transmit data for external analysis may enhance the versatility and utility of the EEG headband across various applications, from personal health monitoring to clinical diagnostics and scientific research.

The main band 12 may also define a receptacle for receiving a grounding electrode 16. This grounding electrode placement 16 may be designed to contact a mastoid bone when the headband is worn, providing a reference point for the EEG measurements. Like the dry EEG electrodes, the ground electrode is connected to the wiring component 15.

In some implementations, the EEG headband may utilize alternative reference points for the grounding electrode beyond the mastoid bone. For instance, the grounding electrode may be positioned to contact the earlobe, which may provide a convenient and accessible location for establishing a reference point. In other aspects, the grounding electrode may be designed to make contact with the forehead, potentially offering a stable reference point while maintaining the discreet profile of the headband.

The grounding electrode may also be configured to contact the nape of the neck in some cases, which may allow for a secure connection while keeping the electrode hidden from view. In other implementations, the grounding electrode may be positioned to make contact with the skin behind the ear, potentially providing a balance between accessibility and concealment.

In some aspects, the EEG headband may incorporate multiple grounding electrode locations, allowing users or clinicians to select the most suitable reference point based on individual anatomy or specific monitoring requirements. The headband may also be designed with an adjustable grounding electrode that can be positioned at various points along the main band 12, potentially enhancing the versatility of the device across different user populations.

The choice of grounding electrode placement may depend on factors such as signal quality, user comfort, and the specific requirements of the EEG application. In some implementations, the grounding electrode may be designed as a removable component, allowing for easy cleaning or replacement as needed.

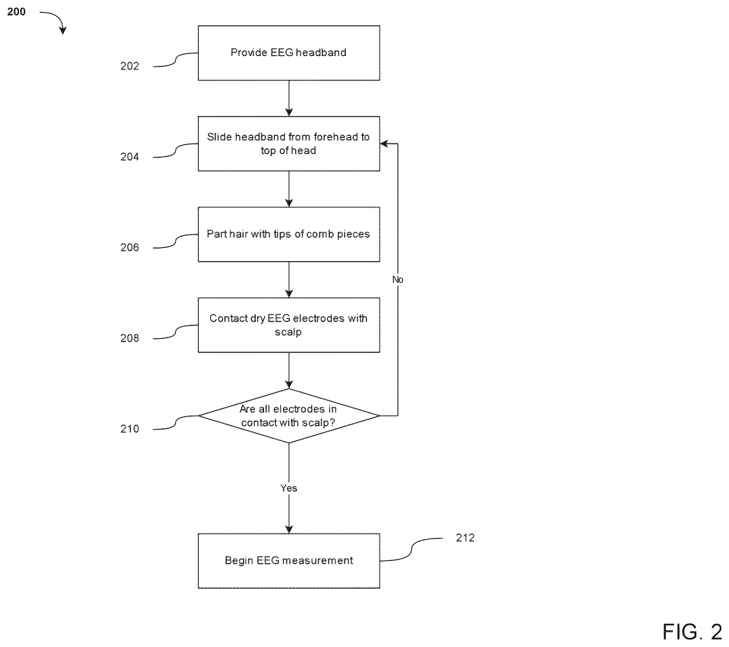

Referring now to FIG. 2, and in brief overview, a method 200 for using the compact and concealed electroencephalography (EEG) headband comprises providing an EEG headband (step 202), sliding the headband from a forehead to a top of a head (step 204), parting hair with tips of the comb pieces as the headband moves backward (step 206), contacting dry EEG electrodes housed in the comb pieces with a scalp (step 208), checking whether all electrodes are in contact with the scalp (step 210), adjusting the headband position if needed (returning to step 204), and beginning EEG measurement (step 212).

Still referring to FIG. 2, and in greater detail, the method 200 begins by providing an EEG headband (step 202), which is slid from the forehead of a subject to the top of the subject's head (step 204).

In some implementations, the EEG headband may be provided in a modular or partially assembled state, allowing for customization and optimization of the device after initial placement on the subject's head. This approach may offer several advantages in terms of fit, comfort, and signal quality.

For instance, the main band 12 may be positioned on the subject's head without the electrodes initially in place. Once the band is properly situated, dry EEG electrodes may be inserted into the electrode receptacles. This sequential assembly process may allow for more precise electrode placement, as the positioning of the main band 12 can be adjusted without interference from pre-installed electrodes. Additionally, this may facilitate the use of different electrode types or sizes based on the specific requirements of the EEG recording or the individual subject's scalp characteristics.

In some cases, the wiring component 15 may be designed as a separate module that can be connected to the main band 12 after it has been positioned on the subject's head. This modular approach to the wiring may offer several benefits. It may allow for easier maintenance and replacement of the electronic components without needing to remove or replace the headband. Furthermore, different wiring configurations or processing modules may be swapped in based on the specific EEG application or research protocol being conducted.

The grounding electrode may also be designed as a separate component that can be attached to the main band 12 after initial placement. This may allow for flexibility in choosing the most appropriate grounding location for each individual subject or specific EEG recording scenario. The grounding electrode may be designed with various attachment mechanisms, such as snap-on connectors or adjustable positioning tracks, to facilitate optimal placement and secure connection.

In some implementations, the stability comb pieces 13 may be designed as removable or adjustable components. This may allow users or clinicians to customize the stability and fit of the headband based on individual head shapes, hair types, or comfort preferences. The ability to add, remove, or reposition stability comb pieces 13 after the main band 12 is in place may contribute to improved electrode contact and overall device stability.

The modular or partially assembled approach may also extend to the comb pieces 11. In some aspects, these pieces may be designed to be interchangeable or adjustable, allowing for customization of the electrode layout or density based on the specific requirements of the EEG recording. This flexibility may enable the headband to be adapted for various applications, from standard clinical EEG recordings to more specialized research protocols.

In some cases, the headband may incorporate a system for fine-tuning electrode pressure or position after the initial placement. This may involve adjustable tensioning mechanisms within the main band 12 or individual electrode mounts that can be manipulated to optimize scalp contact without removing the headband.

The ability to assemble or adjust components of the EEG headband after initial placement may also facilitate the integration of additional sensors or monitoring devices. For example, accelerometers for motion tracking, pulse oximeters for simultaneous monitoring of blood oxygen levels, or temperature sensors may be added to the headband setup based on the specific requirements of the monitoring protocol.

This modular or partially assembled approach to the EEG headband may enhance its versatility and usability across a wide range of applications and user populations. It may allow for more precise customization of the device to individual subject characteristics and specific EEG recording requirements, potentially improving both the quality of the acquired data and the comfort of the wearer during extended monitoring sessions.

The subject's hair is parted (step 206) by the tips of the comb pieces as the headband is slid to the top of the subject's head. This parting of the hair may allow for better contact between the scalp and the electrodes.

Once the headband is in place, the dry EEG electrodes housed in the comb pieces make contact with the scalp (step 208). This contact between the dry EEG electrodes and the scalp may help obtain accurate EEG readings. In some implementations, the electrodes may utilize different approaches to establish and maintain contact with the scalp.

For instance, the electrodes may incorporate spring-loaded mechanisms that allow them to adjust to the contours of the head, potentially providing consistent pressure and contact across various scalp locations. In other cases, the electrodes may feature a flexible or semi-flexible design that conforms to the scalp surface, potentially improving contact stability during movement.

Some implementations may use electrodes with multiple contact points or a brush-like structure, which may help navigate through hair and establish contact with the scalp. These multi-point electrodes may increase the likelihood of maintaining a stable connection even if some contact points are temporarily disrupted.

In certain aspects, the electrodes may be coated with conductive materials that enhance signal transmission while remaining comfortable for extended wear. Some designs may incorporate micro-texturing on the electrode surface to increase the contact area with the scalp without causing discomfort.

The headband may also employ active electrode technologies that amplify the EEG signal at the electrode site, potentially reducing the impact of movement artifacts and improving signal quality even with less than ideal scalp contact.

While sliding the headband from the forehead to the top of the subject's head may be a common application method, it may not be necessary or optimal in all cases. The positioning of the headband may vary depending on the specific EEG monitoring requirements, the subject's head shape, hair type, and personal comfort preferences.

In some implementations, the headband may be designed to sit lower on the forehead, potentially providing easier access to frontal lobe activity. Other designs may allow for placement primarily around the temporal and parietal regions, bypassing the forehead entirely.

For subjects with certain hairstyles or head shapes, a side-to-side application method may be more effective, starting from one ear and moving across to the other. This approach may help in navigating through thick or long hair while ensuring proper electrode placement.

In some cases, the headband may be applied in sections, with users securing one portion of the band before adjusting and securing other sections. This method may allow for more precise positioning of individual electrodes and may be particularly useful for headbands with a larger number of measurement points.

The flexibility in application methods may enhance the usability of the EEG headband across diverse user populations and monitoring scenarios, potentially improving both the comfort of the wearer and the quality of the EEG data collected.

In some cases, the method 200 includes a decision point at a step 210. The step 210 may check whether all electrodes are in contact with the scalp. If the answer is negative, the method 200 may return to the step 204 to re-adjust the headband position. If the answer is affirmative, the method 200 may proceed to a step 212, where EEG measurement begins.

If all electrodes are in contact with the scalp, the method 200 proceeds to a step 212 where EEG measurement begins. In some cases, a processor embedded in the main band 12 may analyze the EEG data collected by the dry EEG electrodes. The analyzing may comprise detecting generalized seizures based on the collected EEG data.

In some implementations, the EEG headband may incorporate an amplifier and transmission circuit to facilitate the upload of EEG data to cloud-based systems for analysis. This approach may allow for more advanced processing and analysis capabilities beyond what may be feasible with onboard processors alone.

The amplifier may be designed to boost the weak EEG signals collected by the dry electrodes, potentially improving the signal-to-noise ratio and overall data quality. In some cases, the amplifier may be integrated directly into the electrode units, allowing for signal amplification as close to the source as possible. This configuration may help minimize interference and signal degradation that could occur during transmission through the headband's wiring.

The transmission circuit may utilize various wireless communication protocols to upload the amplified EEG data to cloud servers. In some implementations, the headband may incorporate Bluetooth Low Energy (BLE) technology for short-range transmission to a nearby smartphone or tablet. The mobile device may then act as a relay, sending the data to cloud servers via cellular or Wi-Fi networks. In other cases, the headband may include built-in cellular or Wi-Fi capabilities, allowing for direct data transmission without the need for an intermediary device.

Once the EEG data is uploaded to the cloud, it may undergo various analysis processes. In some aspects, the cloud-based analysis may employ advanced machine learning algorithms to detect patterns indicative of generalized seizures. These algorithms may be trained on large datasets of EEG recordings from diverse patient populations, potentially improving their accuracy and reliability in identifying seizure activity.

The cloud-based analysis may also focus on detecting silent seizures, which may not manifest with obvious physical symptoms but can still have significant impacts on brain function. In some implementations, the analysis algorithms may look for subtle changes in EEG patterns that may be characteristic of these non-convulsive seizures. The increased computational power available in cloud environments may allow for more complex and nuanced analysis techniques that could be particularly effective in identifying these less obvious seizure events.

In some cases, the cloud-based system may provide real-time analysis and alerts. As EEG data is continuously uploaded, the system may process the information and send notifications to healthcare providers or caregivers if seizure activity is detected. This real-time monitoring capability may be particularly valuable for patients at risk of status epilepticus or other severe seizure events that require immediate intervention.

The cloud-based approach may also facilitate long-term data storage and trend analysis. In some implementations, the system may track changes in seizure frequency, duration, or characteristics over extended periods, potentially providing valuable insights for treatment planning and medication management. The aggregated data may also be used for research purposes, contributing to a better understanding of seizure disorders and the development of more effective interventions.

In some aspects, the cloud-based analysis system may incorporate adaptive learning capabilities. As it processes more data from individual users, the system may refine its detection algorithms to better account for patient-specific EEG patterns and seizure characteristics. This personalized approach may lead to improved accuracy in seizure detection and reduced false positive rates over time.

The combination of onboard amplification, wireless transmission, and cloud-based analysis may enhance the overall capabilities of the EEG headband system. This approach may allow for more sophisticated data processing and interpretation while maintaining the compact and user-friendly nature of the wearable device itself.

In some implementations, upon detection of a generalized seizure, the processor may transmit an alert. In some implementations, the EEG headband may incorporate various methods for alerting users, caregivers, or medical professionals about detected seizure events. These alert mechanisms may be designed to provide timely and effective notifications while maintaining the discreet nature of the device.

Visual alerts may be integrated into the headband or associated devices. For instance, the headband may include small LED indicators that illuminate or change color upon seizure detection. In some cases, these LEDs may be positioned discreetly on the headband, visible only to the user or caregiver. Alternatively, the visual alerts may be displayed on a connected smartphone, smartwatch, or dedicated monitoring device. These displays may show color-coded warnings, graphical representations of seizure intensity, or text notifications.

Audio alerts may also be employed to notify users or caregivers of detected seizure events. The headband may incorporate a small speaker or buzzer that emits a distinct sound pattern when a seizure is detected. In some implementations, the audio alert may be customizable, allowing users to select from various tones or volumes to suit their preferences and environment. For more discreet notifications, the audio alerts may be transmitted to a paired Bluetooth earpiece or hearing aid, providing private and immediate notification to the user.

Written alerts may be generated and transmitted through various channels. The EEG headband may be connected to a smartphone app that receives and displays text notifications describing the nature and timing of detected seizure events. In some cases, these written alerts may be automatically sent as text messages or emails to designated caregivers or medical professionals. The content of these messages may include details such as seizure duration, intensity, and any pre-programmed instructions for response.

In some implementations, the alert system may utilize a combination of these methods. For example, a detected seizure may trigger a vibration alert on the user's smartwatch, accompanied by a visual display of seizure information and an automated text message to emergency contacts. The system may also be designed to escalate alerts if no response is detected, potentially progressing from discreet notifications to more noticeable alarms.

The choice and configuration of alert methods may be customizable, allowing users to select the most appropriate notification strategies based on their lifestyle, severity of condition, and support network. In some cases, the alert system may be programmed to adapt its notification strategy based on factors such as time of day, user location, or seizure severity, providing context-aware and personalized alerting.

In some implementations, the EEG signals collected by the headband may undergo various post-processing techniques to enhance the quality and interpretability of the data. For instance, the system may employ digital filtering algorithms to remove noise and artifacts from the raw EEG signals. These filters may include notch filters to eliminate power line interference, high-pass filters to remove slow drift, and low-pass filters to attenuate high-frequency noise.

The post-processing may also involve advanced signal processing techniques such as independent component analysis (ICA) or principal component analysis (PCA) to separate and remove artifacts related to eye movements, muscle activity, or other non-neural sources. In some cases, the system may utilize adaptive filtering algorithms that can dynamically adjust to changing signal characteristics or environmental conditions.

Machine learning algorithms may be applied to the post-processed EEG data to identify patterns or features associated with specific neurological events or states. These algorithms may be trained on large datasets of labeled EEG recordings to improve their accuracy in detecting seizures, sleep stages, or other brain states of interest.

In some embodiments, the post-processing may include time-frequency analysis techniques such as wavelet transforms or short-time Fourier transforms. These methods may provide insights into the spectral content of the EEG signals over time, potentially revealing subtle changes in brain activity that may be indicative of impending seizures or other neurological events.

The system may also incorporate contextual information from other sensors integrated into the headband, such as accelerometers or temperature sensors, to enhance the interpretation of the EEG signals. For example, motion data from accelerometers may be used to identify and correct for movement artifacts in the EEG recordings.

In some implementations, the alert system may provide summarized reports of seizure activity and other relevant EEG findings on various timescales. For daily summaries, the system may generate a report at the end of each 24-hour period, detailing the number of detected seizures, their duration, intensity, and timing. These daily reports may also include information on sleep quality, overall EEG patterns, and any notable changes from the user's baseline.

Weekly summaries may provide a broader view of seizure trends and patterns. These reports may include graphical representations of seizure frequency and intensity over the week, highlighting any correlations with external factors such as medication changes, stress levels, or sleep patterns. The weekly summary may also compare the current week's data to previous weeks, potentially identifying longer-term trends or cyclical patterns in seizure activity.

Monthly or quarterly reports may offer an even more comprehensive analysis of the user's EEG data and seizure activity. These summaries may include statistical analyses of seizure frequency, duration, and intensity over the longer period. They may also provide insights into the effectiveness of treatments or lifestyle changes based on observed changes in seizure patterns.

In some cases, the summarization system may allow users or healthcare providers to customize the reporting period and content based on individual needs or clinical requirements. For instance, a user may choose to receive a brief daily update, a more detailed weekly report, and a comprehensive monthly analysis.

The summary reports may incorporate data visualization techniques to make the information more accessible and understandable. This may include heat maps showing the distribution of seizure activity over time, line graphs illustrating trends in seizure frequency or duration, or pie charts breaking down the types of seizures detected.

In some implementations, the summarization system may use natural language processing techniques to generate narrative descriptions of the data, providing plain-language explanations of trends, patterns, and potential areas of concern. These narrative summaries may be particularly helpful for users or caregivers who may find it challenging to interpret raw data or complex visualizations.

The system may also provide comparative analyses in the summary reports, benchmarking the user's seizure activity against population averages or against the user's own historical data. This feature may help users and healthcare providers contextualize the EEG findings and make more informed decisions about treatment or lifestyle modifications.

In some embodiments, the summarization system may integrate with electronic health records or other medical management systems, allowing for seamless sharing of EEG data and seizure summaries with healthcare providers. This integration may facilitate more comprehensive and data-driven clinical decision-making.

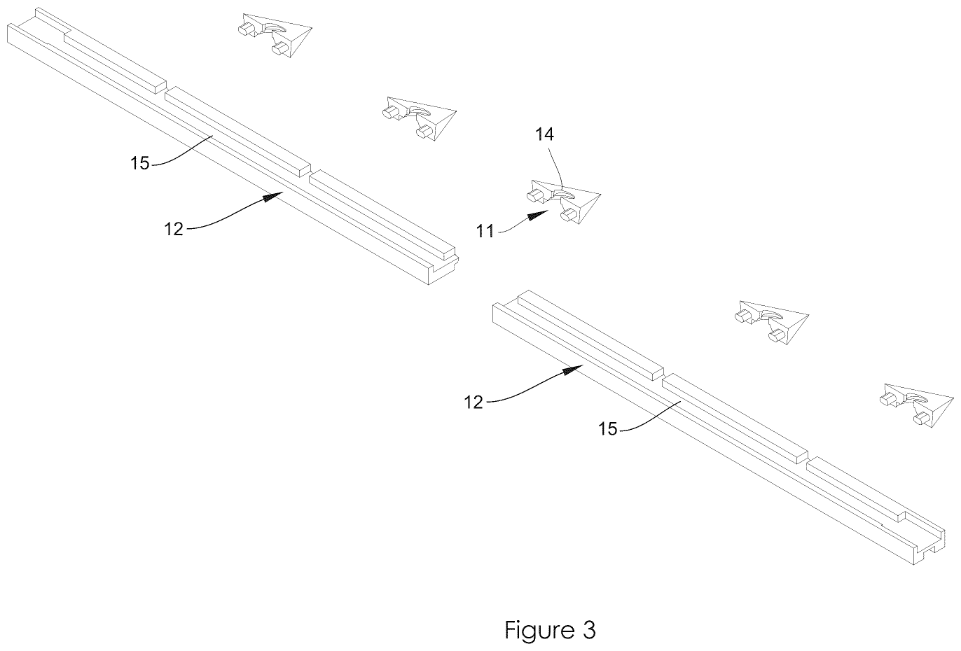

Referring now to FIG. 3, and in brief overview, an exploded view of components of an embodiment of the EEG headband is illustrated. As illustrated in FIG. 3, the main band 12 may be formed from disparate sections. These main band 12 sections may be identical in design. As shown in FIG. 3, each main band 12 section may have a channel or groove running along its length. In some embodiments, the channel accommodates the wiring component 15. Multiple measurement comb pieces 11 may be arranged between the two main band 12 sections. Each measurement comb piece 11 may define a receptacle 14 for receiving an electrode. As shown in FIG. 3, each measurement comb piece 11 may have protruding elements to interface with, and connect to, the main band 12 sections.

In some implementations, the main band 12 sections may be connected using a snap-fit mechanism. This approach may involve interlocking protrusions and recesses along the edges of the main band 12 sections, allowing for secure attachment while maintaining the flexibility to disassemble the headband for maintenance or electrode replacement. The main band 12 sections may also be joined using a sliding rail system. In this configuration, one section may feature a protruding rail that slides into a corresponding groove on the adjacent section. This method may provide a secure connection while allowing for adjustability in the overall circumference of the headband to accommodate different head sizes. In other aspects, the main band 12 sections may be connected using flexible hinges or joints. These hinges may be integrated into the ends of each section, allowing for articulation between segments. This approach may enhance the headband's ability to conform to various head shapes while maintaining a secure fit. The connection between main band 12 sections may utilize a magnetic coupling system. Small, embedded magnets in complementary positions on adjacent sections may provide a strong yet easily separable connection. This method may offer quick assembly and disassembly while ensuring a secure fit during use. In some embodiments, the main band 12 sections may be joined using a tongue-and-groove system. One section may feature a protruding tongue that fits into a corresponding groove on the adjacent section. This interlocking design may provide structural integrity while allowing for some flexibility in the overall headband. The main band 12 sections may be connected using a modular link system. Each section may have standardized connection points at its ends, allowing for the addition or removal of sections to customize the headband's size and electrode configuration. This approach may provide versatility in adapting the headband for different monitoring needs or user preferences. In some implementations, the main band 12 sections may be joined using a flexible bridge component. This bridge may be made of a highly flexible material that stretches between and securely attaches to adjacent sections. This method may allow for a customizable fit while maintaining electrical continuity for the embedded wiring component 15.

The connection between main band 12 sections may also incorporate a locking clasp mechanism. This may involve a hinged clasp on one section that securely fastens to a receiving component on the adjacent section. The locking clasp may provide a reliable connection while allowing for easy donning and doffing of the headband. In some other aspects, the main band 12 sections may be connected using a series of small, flexible connectors distributed along their length. These connectors may be made of a durable, elastic material and may attach to designated points on adjacent sections. This distributed connection approach may provide uniform tension and flexibility across the entire headband.

In addition to the protruding elements shown in FIG. 3, the measurement comb pieces 11 may be connected to the main band 12 sections using various other methods. For instance, the measurement comb pieces 11 may incorporate a slide-and-lock mechanism, where each piece slides into a corresponding slot on the main band 12 sections and locks into place. This approach may allow for easy insertion and removal of the measurement comb pieces 11 while providing a secure connection during use.

In some implementations, the measurement comb pieces 11 may feature a twist-lock design. The base of each comb piece may have a cylindrical protrusion with locking tabs that can be inserted into a matching receptacle on the main band 12 sections. A quarter-turn rotation may then secure the comb piece in place. This method may offer quick installation and removal while ensuring a stable connection.

The connection between the measurement comb pieces 11 and the main band 12 sections may also utilize a magnetic attachment system. Small, powerful magnets may be embedded in both the comb pieces and the main band sections, allowing for rapid and precise positioning. This approach may provide flexibility in adjusting the placement of the comb pieces while maintaining a secure hold.

In some aspects, the measurement comb pieces 11 may be attached to the main band 12 sections using a flexible, snap-on mechanism. The base of each comb piece may feature flexible tabs that snap into corresponding recesses on the main band sections. This design may allow for some movement and adjustment of the comb pieces while keeping them securely attached.

The measurement comb pieces 11 may also be connected to the main band 12 sections using a rail system. The base of each comb piece may have a groove that slides onto a raised rail on the main band sections. This method may allow for easy adjustment of the comb pieces' positions along the length of the headband.

In some implementations, the connection may involve a bayonet mount system, similar to those used in camera lens attachments. The base of the measurement comb pieces 11 may have pins that align with L-shaped slots on the main band 12 sections. Inserting and twisting the comb piece may lock it securely in place.

The attachment method may also incorporate a threaded connection system. The base of each measurement comb piece 11 may feature fine threads that screw into corresponding threaded receptacles on the main band 12 sections. This approach may provide a very secure connection and allow for precise adjustment of the comb pieces' orientation.

In some aspects, the connection between the measurement comb pieces 11 and the main band 12 sections may utilize a push-button release mechanism. Each comb piece may have a spring-loaded button that, when pressed, allows the piece to be inserted into or removed from a slot on the main band sections. This design may offer quick and easy adjustment while maintaining a secure hold during use.

The measurement comb pieces 11 may also be attached using a flexible, adhesive-backed hook-and-loop fastener system. This method may allow for easy repositioning of the comb pieces and may be particularly useful for customizing the headband configuration to individual users' needs.

In some implementations, the connection may involve a dovetail joint system. The base of each measurement comb piece 11 may have a tapered, wedge-shaped protrusion that slides into a corresponding tapered groove on the main band 12 sections. This method may provide a secure, self-tightening connection that resists movement during use.

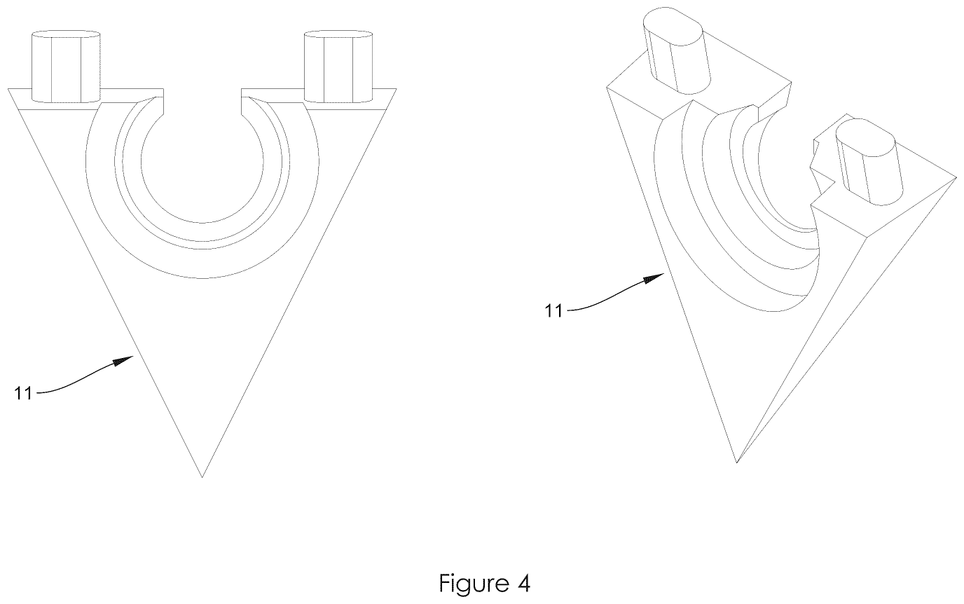

Turning to FIG. 4, two perspective views of a triangular comb piece component of the EEG headband are shown. The left view presents a front-facing perspective while the right view offers an angled perspective of the same component. The comb piece features a triangular base that tapers to a point at the bottom. At the top of the triangular base, there may be cylindrical protrusions designed to interface and connect with the main band. There may be cutouts in the front center of the triangular comb designed to house the electrodes. This design may integrate both structural and functional elements—the triangular shape may allow for hair parting functionality while providing a stable base for the electrode housing. The right perspective view provides additional detail of how the cutouts may be designed to adjust the protrusion distance of the electrode relative to the comb. This arrangement may allow for proper electrode contact with the scalp while the triangular base manages hair positioning. The overall structure demonstrates a compact and integrated design that may combine the electrode mounting capability with the hair management functionality in a single component.

The descriptions of the various embodiments of the present disclosure have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Accordingly, other implementations are within the scope of the following claims.

1. An electroencephalography (EEG) headband comprising:
a main band spanning across frontal, parietal, and temporal lobes of a brain;
a plurality of comb pieces located along the main band, each comb piece comprising a triangular shape and housing a dry EEG electrode; and
a grounding electrode located on the main band, the grounding electrode contacting a mastoid bone.

2. The electroencephalography (EEG) headband of claim 1, the main band comprising a flexible material.

3. The electroencephalography (EEG) headband of claim 1, the plurality of comb pieces comprising stability comb pieces located along the main band to hold the main band flush against a scalp.

4. The electroencephalography (EEG) headband of claim 1, the dry EEG electrodes positioned at T3, C3, Cz, T4, and C4 locations according to a standard 10-20 EEG system.

5. The electroencephalography (EEG) headband of claim 1, further comprising electronics and wiring embedded in the main band.

6. The electroencephalography (EEG) headband of claim 1, the comb pieces pointing away from a forehead when the main band is worn.

7. The electroencephalography (EEG) headband of claim 5, the electronics comprising a processor for analyzing EEG data collected by the dry EEG electrodes.

8. A method of using an electroencephalography (EEG) headband, the method comprising:
sliding the headband from a forehead to a top of a head, the headband comprising a main band and a plurality of comb pieces located along a main band;
parting hair with tips of the comb pieces as the headband moves backward; and
contacting dry EEG electrodes housed in the comb pieces with a scalp.

9. The method of claim 8, the main band comprising a flexible material.

10. The method of claim 8, the plurality of comb pieces comprising stability comb pieces located along the main band to hold the headband flush against the scalp.

11. The method of claim 8, the dry EEG electrodes positioned at T3, C3, Cz, T4, and C4 locations according to a standard 10-20 EEG system.

12. The method of claim 8, further comprising transmitting collected EEG data for analysis.

13. The method of claim 12, the analyzing comprising detecting generalized seizures based on the collected EEG data.

14. The method of claim 13, further comprising transmitting an alert upon detection of a generalized seizure.

15. An electroencephalography (EEG) system comprising:
a headband comprising a main band and a plurality of comb pieces located along the main band;
dry EEG electrodes housed in the comb pieces;
a grounding electrode located on the main band; and
wiring embedded in the main band connecting the dry EEG electrodes and the grounding electrode.

16. The electroencephalography (EEG) system of claim 15, the main band comprising a flexible material.

17. The electroencephalography (EEG) system of claim 15, the plurality of comb pieces comprising stability comb pieces located along the main band to hold the headband flush against a scalp.

18. The electroencephalography (EEG) system of claim 15, the dry EEG electrodes positioned at T3, C3, Cz, T4, and C4 locations according to a standard 10-20 EEG system.

19. The electroencephalography (EEG) system of claim 15, further comprising a processor embedded in the main band, the processor analyzing EEG data collected by the dry EEG electrodes.

20. The electroencephalography (EEG) system of claim 19, the processor detecting generalized seizures based on the collected EEG data.
